(19) United States
(12) Patent Application Publication
(10) Pub. No.: US 2018/0318823 A1
(43) Pub. Date: Nov. 8, 2018

(54) Laboratory Cabinet

(71) Applicant: Binder GmbH, Tuttlingen (DE)

(72) Inventors: Eugen Baumgaertner, Villingen-Schwenningen (DE); Patrick Efinger, Balgheim (DE); Ewald Storz, Rietheim-Weilheim (DE); Hartmut Winkler, Tuttlingen (DE); Richard Leidolt, Singen (DE); Peter M. Binder, Altnau (CH)

(73) Assignee: Binder GmbH, Tuttlingen (DE)

(21) Appl. No.: 15/965,513

(22) Filed: Apr. 27, 2018

(30) Foreign Application Priority Data
Apr. 28, 2017 (DE) 10 2017 109 263.3

(51) Int. Cl. B01L 1/02; C12M 1/00; B01L 7/00; E05B 65/00

(52) U.S. Cl. CPC B01L 1/025; E05B 65/0042; B01L 7/00; C12M 41/14

(57) ABSTRACT

A laboratory cabinet, for example a cold room, heating cabinet, drying oven or incubator, with a housing having at least one outer door with an outer door closure, which has at least one inner chamber, which is closable by an inner door with an inner door closure, wherein the inner door closure is a mechanical closure, that the laboratory cabinet with the outer door closed has actuatable first means for releasing the inner door closure of the inner door, which are electrically actuatable, and that the laboratory cabinet has second means which are designed such that upon opening the outer door, the inner door also opens.

### CROSS-REFERENCE TO RELATED APPLICATIONS

This application claims priority to German Patent Application No. 10 2017 109 263.3, filed Apr. 28, 2017, which is incorporated by reference in its entirety.

### BACKGROUND

The application relates to a laboratory cabinet having the features and structures disclosed herein.

### SUMMARY

Laboratory cabinets with a housing having at least one outer door with an outer door closure and having at least one inner chamber. Such laboratory cabinets often have fittings with which a defined temperature and/or defined climatic conditions, for example a defined humidity, can be set in the interior space. For example, cold rooms, heating cabinets, drying ovens or incubators are known. To prevent the climatic conditions in the inner chamber from being disturbed when the outer door is opened, it is known that the inner chamber may be closed by an inner door with an inner door closure. The outer door serves essentially for preventing air exchange between the inner chamber of the laboratory cabinet and the environment. The inner door prevents exchange of air from the inner chamber with the environment when the outer door is opened. The inner door can be made of transparent material, which makes it possible to look into the inner chamber when the outer door is opened without disturbing the climatic conditions in the inner chamber.

Also known are laboratory cabinets in which the inner chamber is divided into several partial chambers and several inner doors close the smaller partial chambers, so that the climate in the other partial chambers is disturbed as little as possible when sample material is placed in one of the partial chambers or removed from the partial chamber.

In the known laboratory cabinets, it is necessary for a user first to open the outer door of the laboratory cabinet and then it is possible to open the inner door with a second handle.

The problem of the present disclosure consists of further developing a laboratory cabinet so that it can be handled in a more user-friendly manner and especially the opening of the inner door is simplified.

The problem of the present disclosure is solved by a laboratory cabinet with the features and structures disclosed herein.

Advantageous embodiments and further developments of the present disclosure are presented in the dependent claims.

The laboratory cabinet according to the present disclosure, for example cold room, heating cabinet, drying oven or incubator, with a housing having an outer door with an outer door closure, which has at least one inner chamber closable with an inner door with an inner door closure, is characterized in that the inner door closure is a mechanical closure, that the laboratory cabinet when the outer door is closed has operable first means for releasing the inner door closure of the inner door, said means being electrically actuatable, and that the laboratory cabinet has second means which are designed such that when the outer door is opened, the inner door likewise opens. A laboratory cabinet of such a design makes it possible for the user to release the inner door even before the outer door is opened, and means that when the outer door is opened, the inner door is already opened, so that the additional handle, with which the inner door must be separately opened after opening the outer door is opened can be omitted.

In the present case a closure is to be defined as the mechanism that secures the corresponding doors against accidentally swiveling. An additional locking mechanism may be provided on any of the closures to prevent the closure from being opened.

According to an advantageous embodiment of the present disclosure, the mechanical closure has a permanent magnet, a latching mechanism, a spring mechanism, a snap mechanism and/or a ball catch mechanism and the like. Closures of these types are not very interference-prone, especially at high or low temperatures or high humidity.

A preferred embodiment of the present disclosure provides that the first means is designed as an electromagnet disposed on the outer door or the inner door, which in the actuated state interacts with the other, outer door or inner door or with a magnetizable element disposed on the other, outer door or inner door, and which in the actuated state cooperates with the other, outer door or inner door or with a magnetizable element disposed on the other, outer door or inner door and the holding force of which is greater than the closing force of the mechanical closure. In other words, advantageously the first means is designed as an electromagnet, which is disposed on either the outer door or the inner door. In the actuated state the electromagnet, when it is disposed on the outer door, cooperates with the inner door or a magnetizable element disposed on the inner door, whereas when it is disposed on the inner door, it cooperates with the outer door or a magnetizable element disposed on the outer door. In this way the electromagnet creates a coupling between the outer door and the inner door. Because the holding force of the electromagnet is greater than the closing force of the mechanical closure, the mechanical closure of the inner door opens against the closing force when the outer door opens, and thus it releases. If the electromagnet is also energized during the further opening process of the outer door, the inner door will follow the outer door, attracted by the electromagnet, so that in this case the electromagnet can not only form the electrically actuatable first means for releasing the inner door closure of the inner door, but at the same time also can form the second means.

According to an advantageous embodiment of the present disclosure, the first means are designed especially as electromagnets disposed on either the outer door or inner door, which in the actuated state releases the mechanical closure. An electromagnet is not very interference-prone under extreme climate conditions.

Advantageously, the electromagnet in the actuated state releases the mechanical closure by moving an element of the mechanical closure, for example by shifting a latch and/or shifting or releasing a snap element. A release of this type is not very interference-prone, particularly at extreme temperatures, especially at extremely low temperatures.

According to an advantageous further development of the present disclosure, the mechanical closure has a snap closure with a spring-loaded protuberance, wherein in the actuated state the electromagnet draws back the den protuberance especially via a lever mechanism, for example a lever linkage, against the spring force, to release the snap closure. A release of this type is not very interference-prone, especially at extreme temperatures, particularly at extremely low temperatures.

An advantageous further development of the present disclosure provides that the first means are designed as magnetically energizable push rods and the mechanical closure has a latching mechanism with a spring-loaded latch with an opening in which a locking head engages, wherein the electromagnet in the actuated state moves the latch against the spring force to release the locking closure. A release of this type is not very interference-prone.

According to a preferred embodiment of the present disclosure, the first means are designed as an electrically extendable pusher or latch or electromagnetically energizable push rod disposed on either the outer door or inner door, which in the extended state engages with an element disposed on the other, outer or inner door. In this way, coupling can be achieved between the inner door and the outer door, which can overcome the closing force of the inner door closure.

The second means according to a preferred embodiment of the present disclosure are designed as followers disposed between the outer door and the inner door. Here, a follower produces coupling between the outer door and inner door, resulting in the fact that upon opening the outer door, the inner door is carried along, especially pulled along.

According to a preferred embodiment of the present disclosure, the follower is designed as a permanent magnet or electromagnet disposed on either the outer door or inner door, which cooperates with the other outer door or inner door or with a magnetizable element disposed on the other outer door or inner door. With a follower designed in this way, magnetic coupling between the inner door and outer door is achieved, which advantageously also allow a relative movement to take place between the inner door and outer door when the two doors pivot around different pivot points.

A preferred embodiment of the present disclosure provides that the follower is designed as an electrically extendable pusher or latch or an electromagnetically energizable push rod disposed on either the outer door or inner door, which in the extended state engages with an element disposed on the other, outer door or inner door. A follower of this type allows reliable coupling, wherein depending on the design of the element engaged from behind, a relative movement between the outer door and inner door, caused by the arrangement of the pivot points of the doors in different positions, is made possible.

According to an advantageous embodiment of the present disclosure, the second means are designed as springs, for example torsion springs, acting on the inner door. Second means designed in this way mean that upon opening of the outer door, the inner door pops open by spring force, without necessitating any other coupling between the inner door and outer door. Nevertheless, the inner door and the outer door remain pivotable independently of one another.

According to a particularly preferred embodiment of the present disclosure, the first means simultaneously form the second means. which has the advantage that the smallest possible number of additional components on the laboratory cabinet are required.

Advantageously the inner door closure can be manually released when the outer door opens, which makes it possible for the user to open the inner door even after opening the outer door, or if several inner doors are present, one or more inner doors may be opened additionally.

A particularly preferred embodiment of the present disclosure provides that a damping element, for example a spring element or an elastic element, is disposed on the outer door or the inner door, and in case of a closing movement of the outer door, this comes to rest between the outer door and the inner door. Upon closing the outer door, the damping element advantageously causes an impact on the inner door such that the inner door closure engages again. In this way it is made possible to close the inner door again simultaneously with the outer door without requiring a separate closing process for the inner door alone.

A preferred embodiment of the present disclosure provides that the inner door is made at least partially of transparent material, especially glass. This makes it possible to look into the inner chamber without the inner door being opened and thus the climatic conditions in the inner chamber being disturbed.

An advantageous further development of the present disclosure provides that the laboratory cabinet has several inner doors and the first means are designed such that one or more of the inner doors can be released using the first means. A design of this type allows for a selector on the outside of the housing such that the user can choose before opening the outer door which inner door or inner doors are to be opened simultaneously with the outer door. Advantageously in this process the possibility also exists that after opening the outer door, inner doors that were not selected can additionally be opened manually if this should be necessary.

### BRIEF DESCRIPTION OF THE DRAWINGS

The subject matter of the present application will be discussed in detail based on the following figures. These show

FIG. 1 a perspective view of an embodiment of a laboratory cabinet with a first embodiment of a closure and releases mechanism with the outer door open,

FIG. 2 a perspective view of the laboratory cabinet according to FIG. 1 with the outer door open and the inner door open,

FIG. 3 an additional perspective view of the laboratory cabinet according to FIG. 2,

FIG. 4 a detail of a cross section through another embodiment of a laboratory cabinet with a second embodiment of a closure and release mechanism,

FIG. 5 a detail of a cross section through another embodiment of a laboratory cabinet with a third embodiment of a closure and release mechanism,

FIG. 6 a detail of a cross section through another embodiment of a laboratory cabinet with a fourth embodiment of a closure and release mechanism,

FIG. 7 a detail of a cross section through another embodiment of a laboratory cabinet with a fifth embodiment of a closure and release mechanism,

FIG. 8 a detail of a cross section through another embodiment of a laboratory cabinet with a sixth embodiment of a closure,

FIG. 9 a detail of a cross section through another embodiment of a laboratory cabinet with a seventh embodiment of a closure and release mechanism,

FIG. 10 a longitudinal section through the closure and release mechanism of the laboratory cabinet according to FIG. 1 with closed inner door and closed outer door,

FIG. 11 a perspective view of the inner door closure of the laboratory cabinet according to FIG. 1,

FIG. 12 a rear view of the inner door closure according to FIG. 11,

FIG. 13 a longitudinal section through the inner door closure according to FIG. 11,

FIG. 14 a longitudinal section through the part of the inner door closure mounted on the frame according to FIG. 11 and

FIG. 15 an exploded view of the part of the inner door closure according to FIG. 11 mounted on the frame.

### DETAILED DESCRIPTION

The same reference numbers even in different embodiments designate the same or similar parts. For better understandability, not all reference numbers are used in all figures.

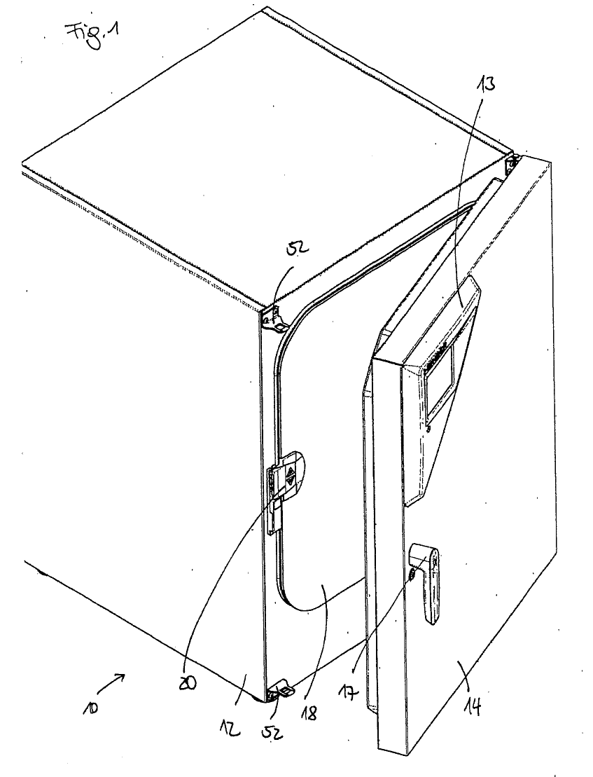

FIGS. 1 to 3 show different perspective views of an embodiment of a laboratory cabinet 10, which for example can be designed as a cold room, heating cabinet, drying oven or incubator. The laboratory cabinet 10 has a housing 12 with an outer door 14 surrounding an inner chamber. The housing 12 may have a bottom side, a top side, two side walls opposite one another and a back wall, wherein an opening opposite the back wall can be closed by the outer door 14. The outer door 14 can be closed by an outer door closure 16, which can be operated using a handle 17. The outer door closure 16 secures the outer door 14 against unintended swiveling. An additional locking mechanism may be provided, which prevents the outer door closure from releasing.

For better thermal insulation, the housing 12 can be designed with double walls. The laboratory cabinet 10 has at least one inner door 18, which closes an opening of the inner chamber facing the outer door 14 to suppress or reduce air exchange between the open inner chamber and the environment when the outer door 14 is opened.

The inner door 18 has an inner door closure 20, which secures the inner door 18 in a closed position against swiveling. In addition, the inner door 18 can have a locking mechanism which prevents the inner door closure 20 from being opened.

The inner door 18 can be made of a transparent material, for example glass.

It is possible for the inner chamber to be subdivided into several partial chambers, each of which is closed by a separate inner door 18, so that only one of the inner doors 18 must be opened when sample material is to be placed in the laboratory cabinet 10, especially in one of the partial chambers, or removed from the laboratory cabinet 10, but the climatic conditions in the other partial chambers are to be disturbed as little as possible.

On the outside of the housing 12 an operating panel 13 can be disposed, with which especially the climatic conditions in the inner chamber of the laboratory cabinet 10, for example the temperature or the humidity, can be set.

Naturally it is basically possible for the laboratory cabinet 10 also to have two or more outer doors 14.

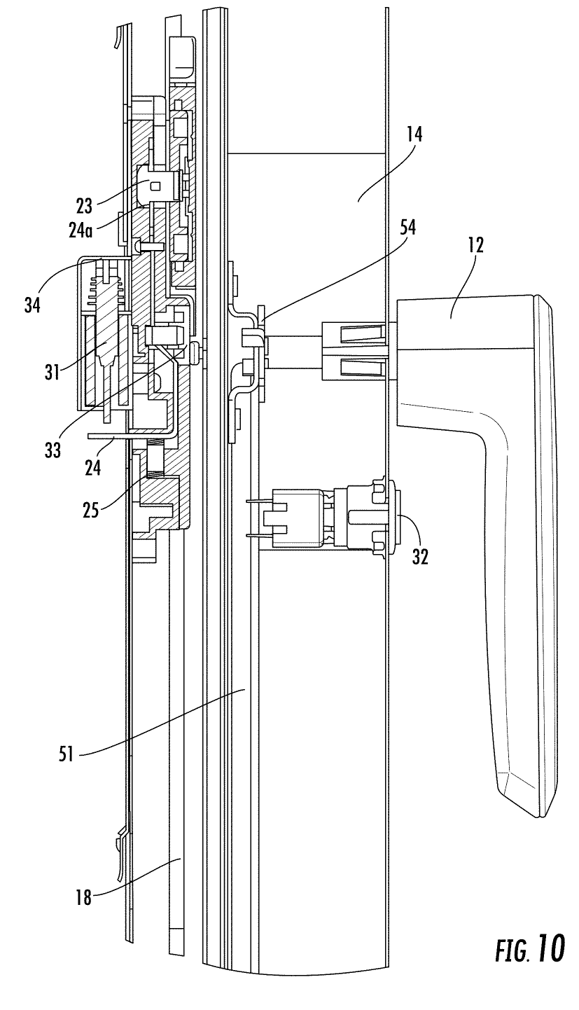

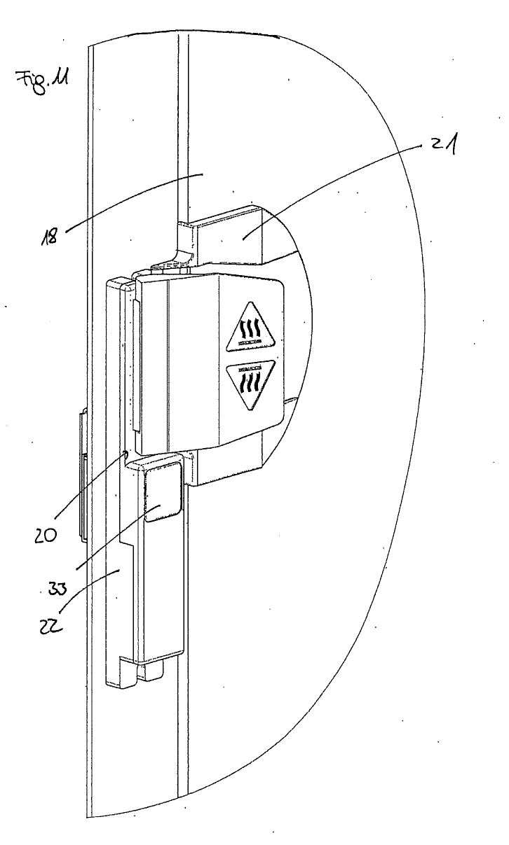

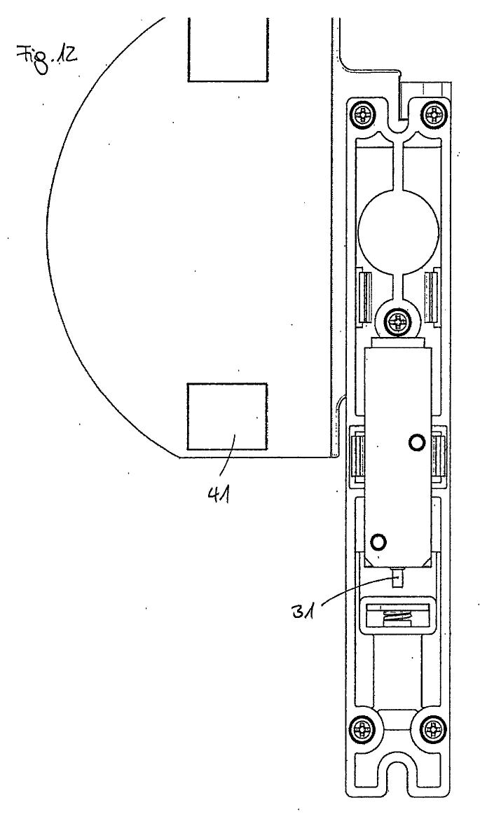

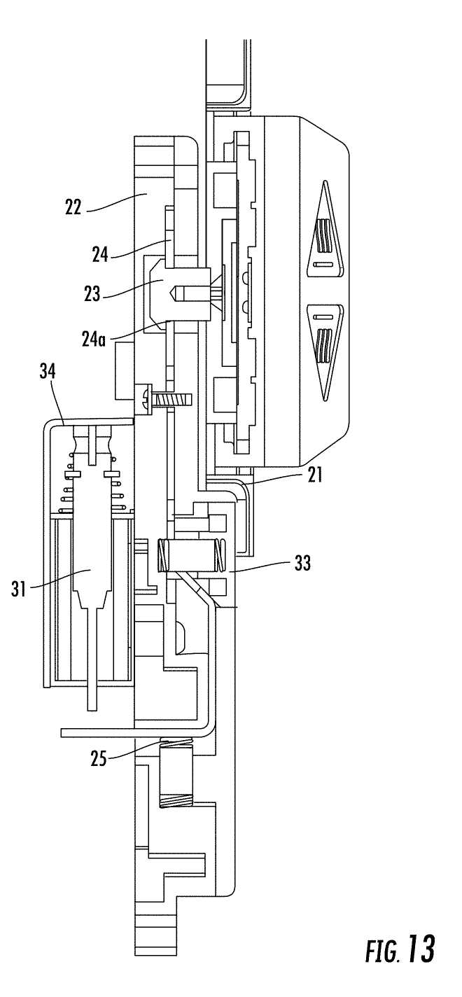

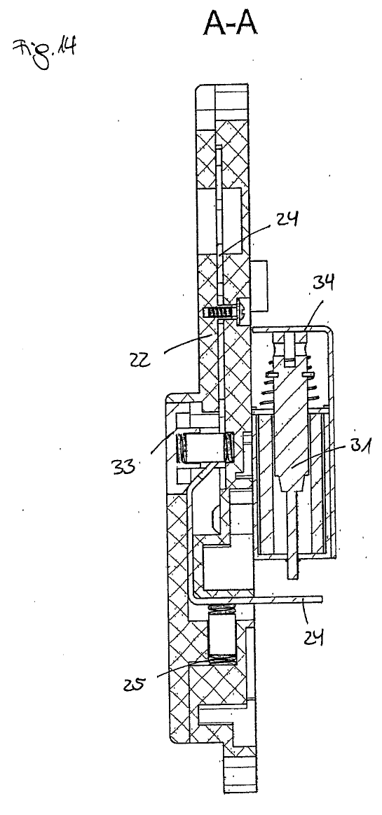

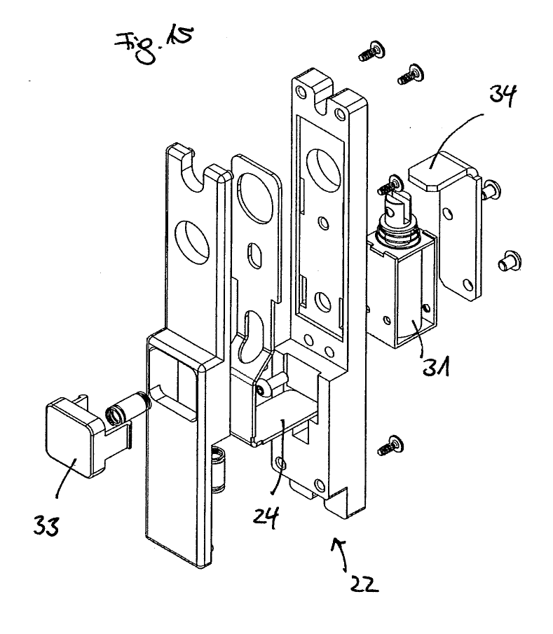

FIGS. 10 to 15 show various detailed views of the closure and release mechanism, especially the inner door closure 20 and the outer door closure 16, of the laboratory cabinet 10 shown in FIGS. 1 to 3.

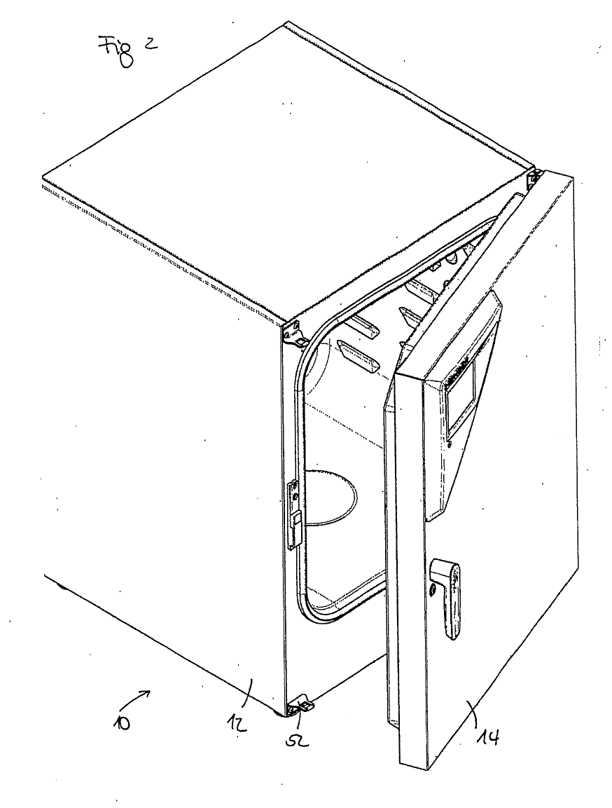

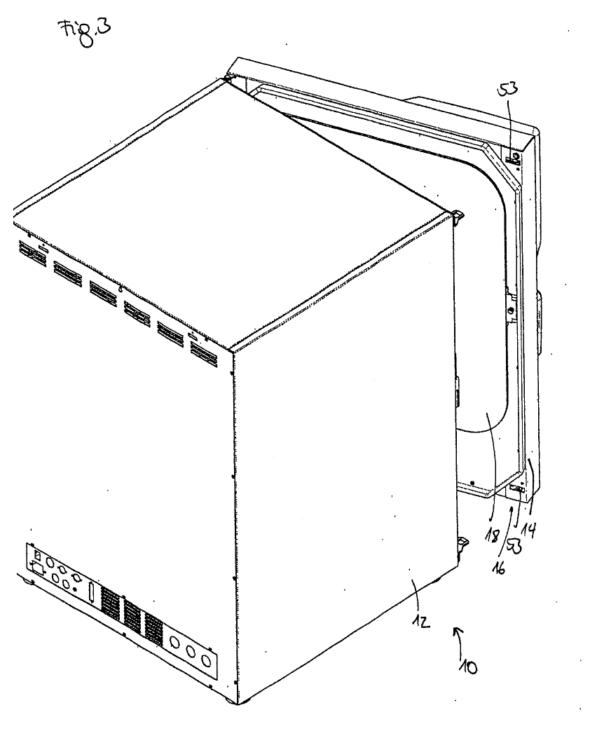

For example, the outer door closure 16 can be designed such that inside of the outer door 14, two vertically positioned rods articulated with an eccentric disk 54, one of which rods 51 is visible in FIG. 10, can be moved apart in the vertical direction when the handle 17 is turned in one direction and can be moved toward one another when the handle 17 is turned in the other direction. If the outer door 14 is closed, two shackles 52 disposed on the front faces of the side walls of the outer door 14, which are especially recognizable in FIGS. 1 and 2, engage with two slits 53 disposed on the inside of outer door 14, which are especially recognizable in FIG. 3. If the handle 17 is then rotated in such a way that the rods 51 are moved apart from one another in the vertical direction, the ends of the rods 51 that are farthest from the handle 17 engage with the shackles 52 and in this way secure the outer door 14 in the closed position against unintended swiveling. When the handles 17 are rotated back the other way, the rods 51 are again moved toward one another and release the 52 shackles, so that the outer doors 14 can again be opened.

The inner door closure 20 is designed as a mechanical closure and can have a latching mechanism. The inner door closure 20 of the embodiment shown in FIGS. 10 to 15 has an inner door closure upper part 21 fastened to the inner door 18 and an inner door closure lower part 22 fastened to the front side of the outer door 14 of the housing 12.

The latching mechanism of the inner door closure 20 can have a locking head 23 disposed on the inner door 18, especially the inner door closure upper part 21, which cooperates with a latch 24 connected to the housing 12, especially to the inner door closure lower part 22, especially by engaging in an opening 24a of the latch 24. The latch 24 is especially spring-loaded by a spring 25. When the inner door 18 is closed, the locking head 23 strikes the latch 24 and moves it against the force of the spring 25 until the locking head 23 engages in the opening 24a of the latch 24.

The laboratory cabinet 10 has first means 30 for releasing the inner door closure 20 of the inner door 18, which are operable when the outer door 14 is closed. The first means 30 are electrically actuatable and can be designed, for example, as magnetically energizable push rods 31. An actuating element 32, with which the power supply can be turned on, is disposed on the outside of the housing 12, for example on the outer door 14, and can for example be disposed in the vicinity of the handles 17 of the outer door 14 or integrated in the operating panel 13. When the power supply is turned on, the push rod 31 is extended and presses against the latch 24, moving this against the force of the spring 25 and releasing the latch closure, especially releasing the locking head 23 from the opening 24a of the latch 24. If the power supply to the push rod 31 is interrupted, the push rod 31 is restored to its position by spring force, especially against a stop 34. Thus, if the user wants to release the inner door 18 as well before opening the outer door 14, the user can actuate the first means 30 with the outer door 14 closed to release the inner door closure 20.

It may be provided for the inner door closure 20 to be released manually even after opening the outer door 14 if the actuating element 32 was not pressed during this process. For this purpose, a pushbutton 33, especially spring-loaded, is disposed on the inner door closure 20, especially on the inner door closure lower part 22. Pressing the pushbutton 33 against the force of the spring acting on the pushbutton 33 causes the latch 24 to move against the spring force of the spring 25 such a distance that the locking head 23 is also released from the opening 24a of the latch 24 and the inner door 18 can be opened.

The laboratory cabinet 10 has second means 40 which are designed such that upon opening the outer door 14, the inner door 18 likewise opens. In the exemplified embodiments shown in FIGS. 10 to 15, the second means 40 are designed as followers disposed between the outer door 14 and the inner door 18, wherein the follower is especially designed as a permanent magnet 41. The permanent magnet 41 can be disposed on the inner door 18 or the outer door 14 and in the present embodiment is disposed on the inner door 18, especially on the inner door closure upper part 21. Since when the outer door 14 is closed, the inner door closure 20 practically touches the outer door 14 or advantageously may also rest in a recess on the inside of the outer door 14, and in addition the inside of the outer door 14 is made of a magnetizable material, or alternatively a magnetizable element is disposed on the inside of the outer door 14, the inner door 18 is magnetically attracted by the outer door 14 magnetically and follows along when the outer door 14 is opened. In particular, the magnetic coupling between the inner door 18 and the outer door 14 can compensate for the relative movement between the inner door 18 and the outer door 14, in that the permanent magnet 41 slides over the inner surface of the outer door 14.

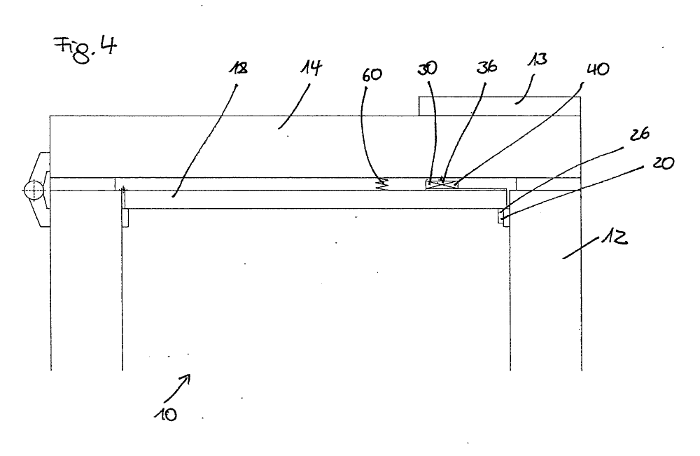

FIG. 4 shows a cross-section through the laboratory cabinet 10 according to FIG. 1 with an alternative embodiment of the closure and release mechanism. The inner door closure 20 in this embodiment is designed as a mechanical closure with a permanent magnet 26.

Instead of a permanent magnet closure, a different mechanical closure may also be selected, which can make the inner door 18 open by exerting a tensile force in the opening direction, for example a closure based on a clamp mechanism, a spring mechanism, a latching mechanism, a snap mechanism and/or a ball catch mechanism.

In this embodiment the first means 30 are designed as an electromagnet 36, which may be disposed either on the inside of the outer door 14 or the outside of the inner door 18. If the electromagnet 36 is disposed on the inner door 18, in the actuated state it can create a magnetic coupling with the outer door 14, if this is made of magnetizable material, or with a magnetizable element disposed on the outer door 14. If the electromagnet 36 is disposed on the outer door 14 it can cooperate correspondingly with the inner door 18 or a magnetizable element disposed on the inner door 18. The electromagnet 36 can be actuated using an actuating element, disposed on the outside of the housing 12, for example disposed on the outerdoor 14 as a separate element or integrated in the operating panel 13. The holding force of the electromagnet 36 in this case is especially greater than the holding force of the permanent magnet closure 26, so that upon opening the outer door 14, the inner door closure 20 will also release and open. In this case the first means 30 designed in this manner as an electromagnet 36 also simultaneously form the second means 40, which cause the inner door 18 also to open upon opening the outer door 14. The relative movement between the outer door 14 and the inner door 18 upon the pivoting of the doors, which is caused by the different pivot points of the two doors 14, 18, can be compensated by the fact that the electromagnet 36 can glide over the smooth door surface. If the electromagnet 36 is disposed on the outer door 14, a cable from the actuating element to the electromagnet 36 which may be required can be laid completely inside the outer door 14 and need not be laid around the pivot point of the inner door 18.

Between the outer door 14 and the inner door 18, preferably on the outer door 14, a damping element 60, for example a spring element or an elastic element, for example a rubber damper, is disposed, which upon closing the outer door 14 comes to rest against the inner door 18 and closes the inner door 18 to the point where the inner door closure 20 latches again.

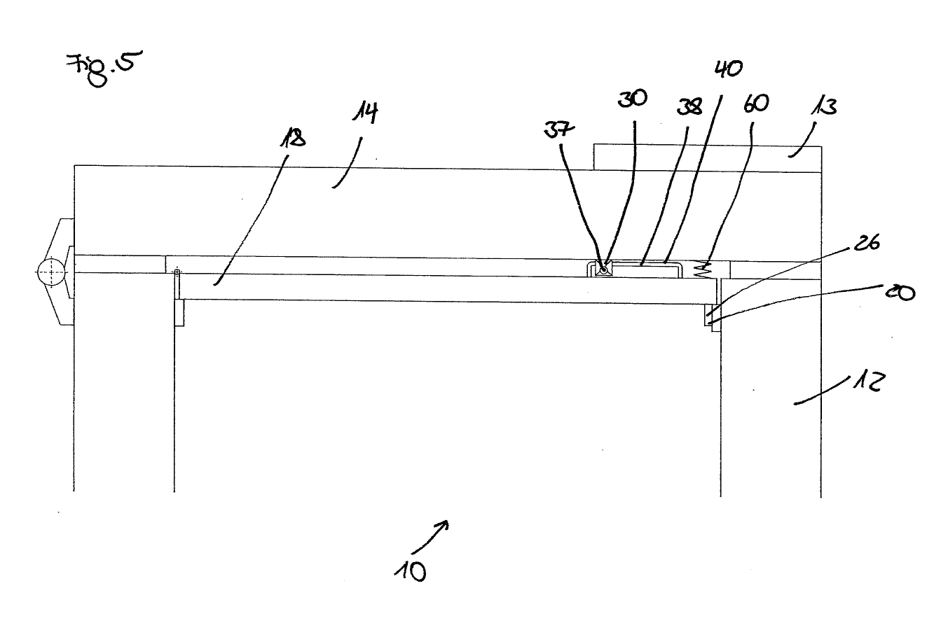

The embodiment of the laboratory cabinet 10 shown in FIG. 5 differs from the laboratory cabinet 10 shown in FIG. 4 only through the design of the first means 30 and the second means 40. The first means 30 are designed as an electrically extendable pusher or latch or electromagnetically energizable push rods 37 disposed on either the outer door 14 or the inner door 18, in the present case especially on the outer door 14. The direction of movement of the push rods 37 here is especially parallel to the axis of rotation of the outer door 14 or the inner door 18. In the extended state, the push rod 37 engages behind an element 38 disposed behind the other of the outer door 14 or inner door 18, in the present case the inner door 18, wherein the element 38 is designed in the manner of a handle or a bracket and forms a stop for the push rods 37 at both ends. When relative movement occurs between the outer door 14 and the inner door 18 upon opening the two doors 14, 18, the push rods 37 can thus glide over a certain distance along the element 38, the distance is nevertheless limited. Once the opening angle of the outer door 14 is reached, at which the push rod 37 strikes one end of the element 38, the outer door 14 can only be opened farther if the push rod 37 is pushed in farther. When the push rod 37 engages behind the element 38, coupling between the outer door 14 and the inner door 18 is achieved, which on one hand releases the inner door closure 20 upon opening the outer door 14, and on the other hand also simultaneously forms the second means 40, which make the inner door 18 also open upon opening the outer door 14.

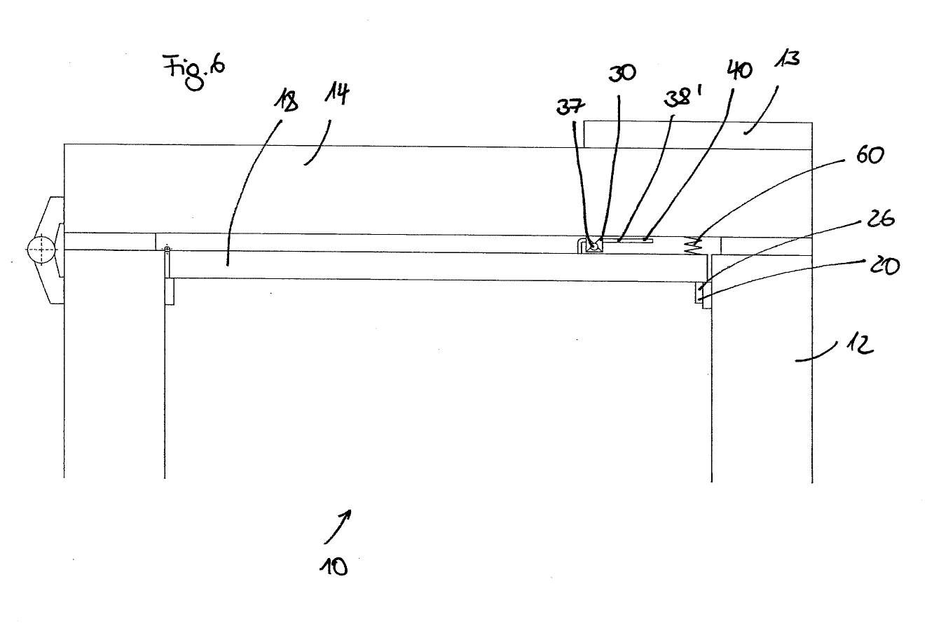

The embodiment of the laboratory cabinet 10 shown in FIG. 6 differs from the laboratory cabinet 10 shown in FIG. 5 only through the design of the first means 30 and the second means 40. Instead of the element 38, which in the embodiment of the laboratory cabinet 10 shown in FIG. 5 has a bracket-like design with two fastening points, in the embodiment shown in FIG. 6 the laboratory cabinet 10 uses an element 38′ which is fastened to the inner door 18 on only one side and is open on the other end. The first means 30 of this design release the inner door closure 20 because the permanent magnet closure 26 is disengaged when the push rod 37 is moved outward and the element 38′ is disengaged from behind, since the inner door closure 20 opens when the outer door 14 is opened. At the same time the first means 30 configured in this way also form the second means 40, since the inner door 18 likewise opens when the outer door 14 is opened. Upon opening the outer door 14, the push rod 37 can glide over a certain opening angle along the element 38′, wherein the die inner door 18 is carried along further. Once a certain opening angle is reached, the push rod 37 glides along on the free end of the element 38′ out of the follower position and releases the inner door 18.

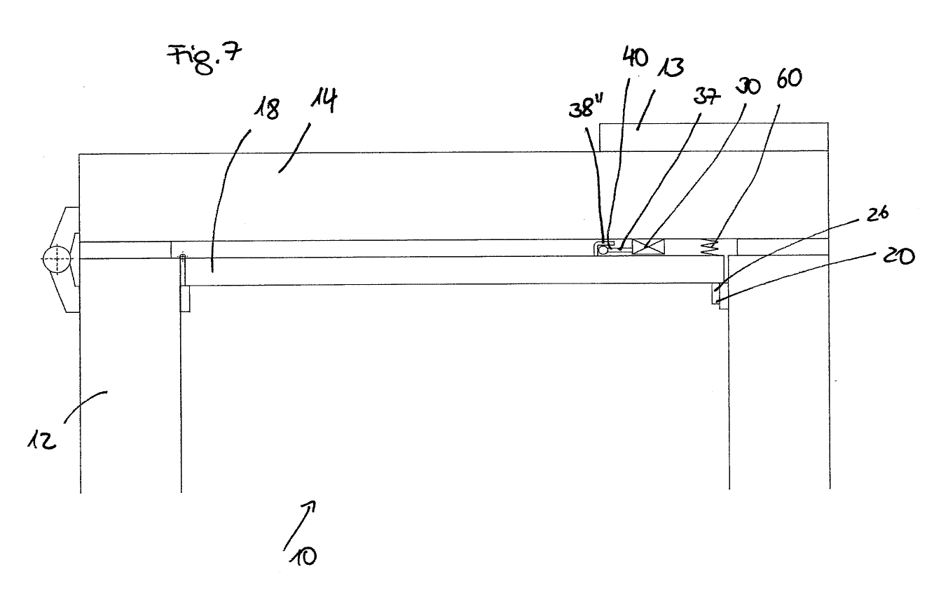

The embodiment of the laboratory cabinet 10 shown in FIG. 7 differs from the embodiment of the laboratory cabinet 10 shown in FIG. 6 only in the design of the first means 30 and the second means 40. The magnetically energizable push rod 37 in the embodiment of the laboratory cabinet 10 shown in FIG. 7 is arranged such that the direction of movement of the push rod 37 is perpendicular to the axis of rotation of the outer door 14 or the inner door 18 and especially parallel to the surface of the inner door 18 and the outer door 14. After activation, the push rod 37 in its extended state can engage with an element 38″ open on one side and in this way form the first means 30 for releasing the inner door closure 20 and the second means 40 for carrying along the inner door 18 upon opening the outer door 14. Because of the relative movement between the outer door 14 and the inner door 18 upon opening the outer door 14, at a certain opening angle the push rod 37 can disengage from the element 38″ and again release the inner door 18.

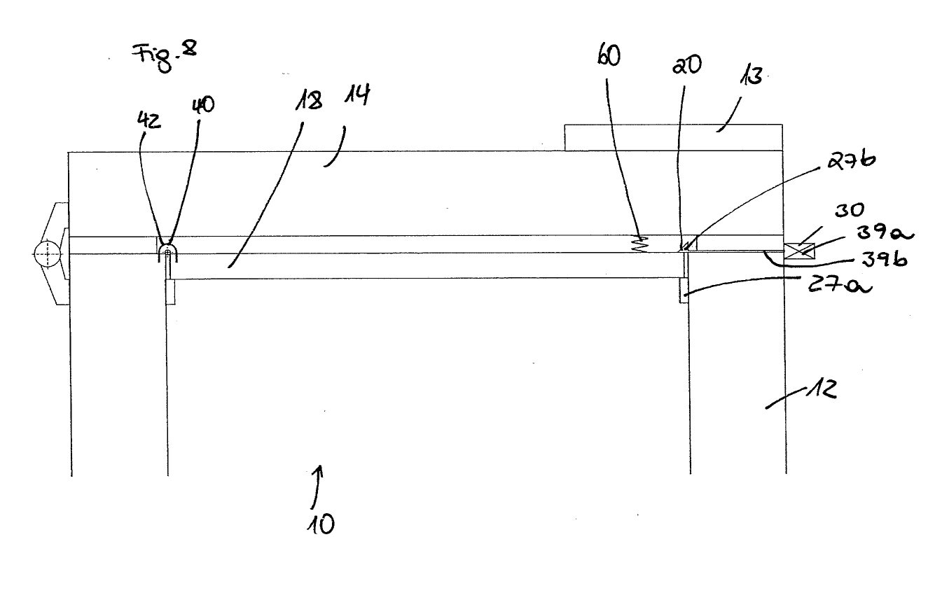

FIG. 8 shows an additional embodiment of laboratory cabinet 10, which differs from the embodiment of the laboratory cabinet 10 shown in FIG. 7 through the design of the inner door closure 20 and the design of the first means 30 and the second means 40.

The inner door closure 20 is designed as a mechanical closure, which has a snap closure with a spring-loaded protuberance 27b. The inner door 18 in the closed position rests against a stop 27a and is locked in this position by the spring-loaded protuberance 27b, which is especially disposed on the housing 12. When the inner door 18 is pressed closed, the protuberance 27b is moved against the spring force, so that the inner door 18 can snap in behind the protuberance 27b.

The first means 30 for releasing the inner door closure 20 is designed as an electromagnet 39a, which can especially be disposed on the outside of the housing 12. The electromagnet 39a is connected over a connecting element 39b, which may be signed for example as a bar or sheet, with the protuberance 27b. The connecting element 39b is preferably passed inward through the sealing gap between the outer door 14 and the front side of a side wall of the housing 12 to the protuberance 27b. The electromagnet 39a can be energized when the outer door 14 is closed, especially fed with current, for example by operating the actuating element 32, as a result of which the connecting element 39b of the protuberance 27b is moved in such a way, especially withdrawn, that the inner door 18 is released. After release of the inner door 18, the protuberance 27b is returned in to its initial position by spring action.

In this embodiment the second means 40 are designed as springs acting upon the inner door 18, for example as torsion springs 42. As soon as the outer door 14 opens and the inner door closure 20 is released, the inner door 18 is acted upon by the torsion spring 22 and forced into an open position.

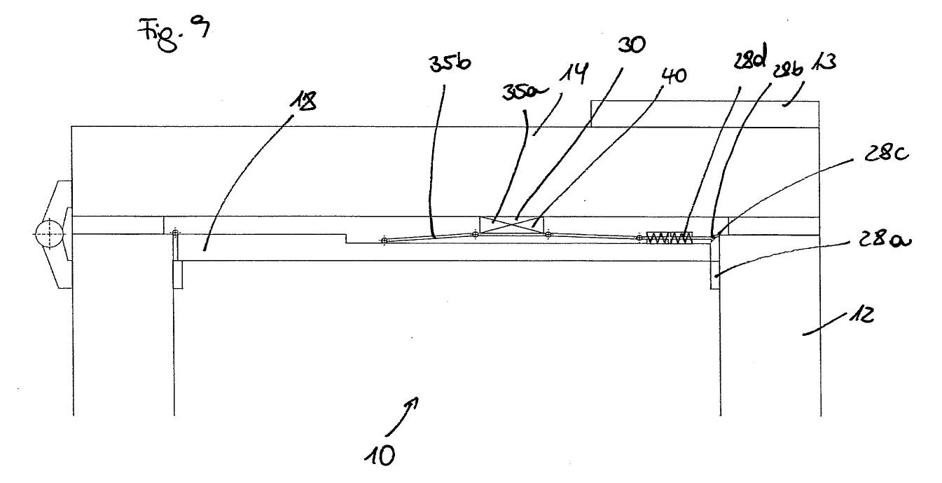

The embodiment of the laboratory cabinet 10 shown in FIG. 9 differs from the embodiment shown in FIG. 8 through the design of the inner door closure 20 and through the design of the first means 30 and the second means 40.

The inner door closure 20 is designed as a mechanical closure, which has a snap closure. The snap closure has a protuberance 28b loaded by a spring 28d, which is disposed on the inner door 18. In the closed position the inner door 18 strikes a protuberance 28c, which is disposed on the inside of the side wall of the housing 12. At a distance from the stop 28a, a protuberance 28c is disposed, which upon closing of the inner door 18 presses the spring-loaded protuberance 28b back against the force of the spring 28d, wherein finally in the closed position the spring-loaded protuberance 28b finally snaps in behind the protuberance 28c.

The first means 30 for releasing the inner door closure 20 have an electromagnet 35a, which is disposed on the inside of the outer door 14. The energization of the electromagnet 35a can take place with the outer door 14 closed, for example by actuating an actuating button disposed on the outside of the housing 12, for example on the outside of the outer door 14 or may also be integrated in the operating panel 13. In the energized state, the electromagnet 35a draws the spring-loaded protuberance 28b back against the force of the spring 28d, especially by acting on a lever mechanism, for example a lever link 35b, which is disposed on the spring-loaded protuberance 28b. The lever link 35b has at least two joints, so that part of the lever link 35b can be moved perpendicular to the plane of the inner door 18, induced by the attractive force of the electromagnet 35a, which results in moving the spring-loaded protuberance 28b parallel to the plane of the inner door 18. In this way the door closure 20 is released by actuating the electromagnet 35a.

However, the first means 30 also simultaneously form the second means 40, which are designed such that upon opening the outer door 14 the inner door 18 likewise opens, since the energized electromagnet 35a produces a magnetic coupling to the lever link 35b and consequently a follower is produced, which causes the inner door 18 to be pulled along when the outer door 14 is opened. Through the joints of the lever link 35b, a relative movement between the outer door 14 and the inner door 18 can also be compensated upon opening the doors 14, 18.

In all the embodiments described, manual opening of the inner door closure 20 is preferably possible if the outer door 14 was opened without releasing the inner door closure 20.

### REFERENCE SYMBOLS

10 Laboratory cabinet
13 Operating panel
12 Housing
14 Outer door
16 Outer door closure
17 Handle
18 Inner door
20 Inner door closure
21 Inner door closure upper part
22 Inner door closure lower part
23 Locking head
24 Latch
24a Opening
25 Spring
26 Permanent magnet
27a Stop
27b Protuberance
28a Stop
28b Spring-loaded protuberance
28c Protuberance
28d Spring
30 First means
31 Push rod
32 Actuating element
33 Pushbutton
34 Stop
35a Electromagnet
35b Lever link
36 Electromagnet
37 Push rod
38 Element
38′ Element
38″ Element
39a Electromagnet
39b Connecting element
40 Second means
41 Permanent magnet
42 Torsion spring
51 Rod
52 Shackle
53 Slit
54 Eccentric disk
60 Damping element

1. A laboratory cabinet, comprising:
a housing;
an outer door with an outer door closure;
an inner chamber;
an inner door with an inner door closure, wherein the inner door closes the inner chamber and the inner door closure is a mechanical closure;
an electrically actuatable device, wherein when the outer door is closed, the electrically actuatable device is operable for releasing the inner door closure of the inner door; and
a follower configured such that when the outer door opens, the inner door likewise opens.

2. The laboratory cabinet according to claim 1, wherein the mechanical closure is one of a permanent magnet, a clamp mechanism, a latching mechanism, a spring mechanism, a snap mechanism, a ball catch mechanism or a combination thereof.

3. The laboratory cabinet according to claim 1, wherein the electrically actuatable device is an electromagnet disposed on either the outer door or the inner door, wherein when the electromagnet is in an actuated state, the electromagnet cooperates with an other door or a magnetizable element disposed on the other door, and generates a holding force greater than a closing force of the mechanical closure.

4. The laboratory cabinet according to claim 1, wherein the electrically actuatable device comprises an electromagnet disposed on either the outer door or inner door, wherein an actuated state the electromagnet unlatches the mechanical closure.

5. The laboratory cabinet according to claim 4, wherein the electromagnet in the actuated state unlatches the mechanical closure by moving an element of the mechanical closure.

6. The laboratory cabinet according to claim 4, wherein the mechanical closure has a snap closure with a spring-loaded protuberance, and the electromagnet in the actuated state draws back the protuberance against the spring force to release the snap closure.

7. The laboratory cabinet according to claim 1, wherein the electrically actuatable device is a magnetically energizable push rod and the mechanical closure has a latching mechanism with a spring-loaded latch having an opening in which a locking head engages, and the push rod an actuated state moves the latch against spring force to release the snap closure.

8. The laboratory cabinet according to claim 1, wherein the electrically actuatable device is a pusher or latch or electromagnetically energizable push rod electrically extendable from the outer door and inner door, which in an extended state engages behind an element disposed on an other door.

9. The laboratory cabinet according to claim 1, wherein the follower is disposed between the outer door and the inner door.

10. The laboratory cabinet according to claim 9, wherein the follower is a permanent magnet or electromagnet disposed on either the outer door or the inner door, wherein the follower cooperates with the outer door or inner door or with a magnetizable element disposed on the outer door or inner door.

11. The laboratory cabinet according to claim 9, wherein the follower is an electromagnetically energizable push rod disposed on either the outer door or inner door, wherein the electromagnetically energizable push rod in an extended state engages behind an element disposed on the outer door or inner door.

12. The laboratory cabinet according to claim 1, wherein the follower is a spring acting upon the inner door.

13. The laboratory cabinet according to claim 1, wherein the electrically actuatable device simultaneously forms the follower.

14. The laboratory cabinet according to claim 1, wherein the inner door closure can be manually unlatched when the outer door is open.

15. The laboratory cabinet according to claim 1, wherein a damping element is disposed on the outer door or the inner door, wherein upon a closing movement of the outer door, the damping element comes to rest between the outer door and the inner door.

16. The laboratory cabinet according to claim 1, wherein the inner door is at least partially made of transparent material.

17. The laboratory cabinet according to claim 1, wherein the laboratory cabinet has several inner doors and that the electrically actuatable device is arranged such that one or more of the inner doors can be unlatched by the electrically actuatable device.

18. A laboratory cabinet, comprising:
a housing;
an outer door that closes the housing;
an outer door closure;
an inner chamber;
an inner door that closes the inner chamber;
an inner door mechanical closure;
an electrically actuatable device that releases the inner door mechanical closure, wherein when the outer door is closed, the electrically actuatable device is operable for releasing the inner door mechanical closure, and wherein the electrically actuatable device is located between the outer door and the inner door;
a follower configured such that when the outer door opens, the inner door opens; and
wherein the follower is located between the outer door and the inner door.
